(19) United States
(12) Patent Application Publication
(10) Pub. No.: US 2026/0354879 A1
(43) Pub. Date: Nov. 12, 2026

(54) SYSTEMS AND METHODS FOR SHAPING IMPLANTS

(71) Applicant: Arthrex, Inc., Naples, FL (US)

(72) Inventors: Devin CONNER, Naples, FL (US); Kevin GALLEN, Naples, FL (US); Charlotte MACDONALD, Naples, FL (US); Dean HUTCHINSON, Naples, FL (US)

(21) Appl. No.: 19/670,928

(22) Filed: May 7, 2026

Related U.S. Application Data
(60) Provisional application No. 63/801,490, filed on May 7, 2025.

(51) Int. Cl. A61B 17/88

(52) U.S. Cl. CPC A61B 17/8863

(57) ABSTRACT

Systems, methods, and kits are used to cut at least a portion of an implant into a tapered shape. The system may include a support shaft passed through a channel of the implant, a collar attached to the support shaft to secure the implant to the support shaft, and a cutting member having a blade and a lumen that receives the implant. The support shaft rotates the implant relative to the blade to form a taper on at least a portion of an outer surface of the implant. The system can include a jig that receives the implant.

### PRIORITY

The patent application claims priority to U.S. Provisional Patent Application No. 63/801,490, filed on May 7, 2025, the entire disclosure of which is incorporated herein.

### TECHNICAL FIELD

The disclosure relates to the field of surgery and, more specifically, to systems and methods of shaping bone implants.

### BACKGROUND

Bone dowels offer surgeons a quick and effective solution for filling bone defects or tunnels during staged revision cases. The use of bone dowels provides an immediate structural and biologic architecture for stability and incorporation.

### SUMMARY

The present inventors recognize that during revision shoulder arthroplasty, removal of existing hardware may result in a cavitary defect in the glenoid. Current options do not fit or conform to specific anatomy of the defect.

Thus, a first aspect of the present disclosure is directed to a system for cutting an implant. The system may include: a support shaft configured to be passed through a channel of the implant; a collar configured to be attached to the support shaft to secure the implant to the support shaft; and a cutting member having a blade and a lumen configured to receive the implant, wherein the support shaft may be configured to rotate the implant relative to the blade to form a taper on at least a portion of an outer surface of the implant.

In some embodiments, the system may include one or more of the following features. The blade may extend at an acute angle relative to a longitudinal axis of the lumen. The acute angle may be from 5° to 15°. The blade may extend tangentially of the lumen. The lumen may have a first opening with a first width and a second opening with a second width, and the first width is greater than the second width. The lumen may have a tapered cross-section. The cutting member may have indicia indicating a width of the lumen. The cutting member may have a housing defining the lumen and an opening that receives the blade, the blade may be fixed to the housing, and an edge of the blade may be exposed to the lumen. The cutting member may have a longitudinal slot between the housing and the blade. The support shaft may have a receiving portion configured to be inserted into the channel of the implant, and a flange proximal of the receiving portion, and the flange may be configured to support a proximal portion of the implant. The collar may be configured to be adjustably attached to the support shaft. The support shaft may have a distal portion with an outer thread, the collar may have an inner thread, and the outer thread may be configured to engage the inner thread to attach the collar to the support shaft. The support shaft may have a proximal portion having a fitting configured to be attached to a tool.

A second aspect of the present disclosure is directed to a kit. The kit may include at least one implant and the system of the first aspect.

In some embodiments, the kit may include one or more of the following features. The at least one implant may include a first implant and a second implant, the first implant may have a first dimension, the second implant may have a second dimension, and the first dimension may be greater than the second dimension. The kit may include a sizing instrument configured to measure a size of a bone defect for the implant. The at least one implant may be an allograft. The at least one implant may be a porous cancellous bone tissue.

A third aspect of the present disclosure is directed to a method. The method may include: attaching an implant to a support shaft; inserting the implant into a cutting member, the cutting member having at least one blade extending at an acute angle relative to a longitudinal axis of the cutting member; and rotating the support shaft relative to the cutting member to form a taper on at least a portion of an outer surface of the implant.

In some embodiments, the method may include one or more of the following features. The method may include advancing the support shaft along the longitudinal axis of the cutting member while rotating the support shaft relative to the cutting member. The taper may be formed substantially uniform around a circumference of the implant. The taper may be formed on less than an entire length of the implant. The attaching of the implant to the support shaft may include inserting the support shaft into a channel of the implant. The method may include attaching a collar onto the support shaft to secure the implant to the support shaft. The attaching of the collar onto the support shaft may include threading the collar onto the support shaft. The method may include attaching the support shaft to a tool, wherein rotating the support shaft uses the tool. The cutting member may include a housing having a lumen defining the longitudinal axis, and the at least one blade may be fixed to the housing. The implant may be an allograft. The implant may be a porous cancellous bone tissue.

A fourth aspect of the present disclosure is directed to a system for cutting an implant. The system may include: a jig having a sleeve configured to receive the implant; and a cutting member having a distal surface with at least one blade, wherein the at least one blade may be configured to be inserted into the sleeve and to be rotated around a longitudinal axis to form a taper on at least a portion of an outer surface of the implant.

In some embodiments, the system may include one or more of the following features. The at least one blade may include a plurality of blades circumferentially spaced around the longitudinal axis. The at least one blade may have an inner surface that is angled relative to the longitudinal axis. The system may include at least one pin, wherein the sleeve of the jig may have at least one hole configured to receive the at least one pin to prevent rotation and/or translation of the implant relative to the jig. The sleeve of the jig may have at least one slot configured to allow for visibility of the implant and/or the at least one blade. The cutting member may have a sleeve configured to receive the sleeve of the jig. The jig may have a proximal surface with a protrusion configured to be received in a channel of the implant. The distal surface of the cutting member may have a protrusion configured to be received in a channel of the implant. The jig may have a shaft extending proximally from the sleeve and a fitting on the shaft configured to be attached to a tool. The cutting member may have a shaft extending distally from the distal surface and a fitting on the shaft configured to be attached to a tool.

A fifth aspect of the present disclosure is directed to a kit. The kit may include at least one implant and the system of the fourth aspect.

In some embodiments, the kit may include one or more of the following features. The at least one implant may include a first implant and a second implant, the first implant may have a first dimension, the second implant may have a second dimension, and the first dimension may be greater than the second dimension. The kit may include a sizing instrument configured to measure a size of a bone defect for the implant. The at least one implant may be an allograft. The at least one implant may be a porous cancellous bone tissue.

A sixth aspect of the present disclosure is directed to a method. The method may include: inserting an implant into a sleeve of a jig; inserting at least one blade of a cutting member into the sleeve of the jig; and rotating the at least one blade around a longitudinal axis relative to the implant to form a taper on at least a portion of an outer surface of the implant.

In some embodiments, the method may include one or more of the following features. Rotating the at least one blade relative to the implant may include rotating a plurality of blades around the longitudinal axis relative to the implant. The method may include inserting the sleeve of the jig in a sleeve of the cutting member. The method may include securing the implant in the sleeve by inserting at least one pin into at least hole in the sleeve of the jig. The method may include inserting a protrusion on a proximal surface of the sleeve in a channel of the implant. The method may include inserting a protrusion on a distal surface of the sleeve in the channel of the implant. The method may include attaching a shaft of the jig to a tool, wherein rotating the at least one blade relative to the implant is with the tool. The method may include attaching a shaft of the cutting member to a tool, wherein rotating the at least one blade relative to the implant is with the tool. The implant may be an allograft. The implant may be a porous cancellous bone tissue.

### BRIEF DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In the drawings:

FIG. 1 illustrates a side view of a first exemplary system for shaping an implant.

FIG. 2 illustrates a side view of the system of FIG. 1 with the implant inserted into a cutting member.

FIG. 3 illustrates an exemplary implant formed by the system of FIGS. 1 and 2.

FIG. 4 illustrates a perspective view of the cutting member of the system of FIGS. 1 and 2.

FIG. 5 illustrates a cross-sectional view of the cutting member of FIG. 4.

FIG. 6 illustrates an exemplary support shaft of the system of FIGS. 1, 2, 4 and 5.

FIG. 7 illustrates a flow chart of a method of using the system of FIGS. 1, 2 and 4-6.

FIGS. 8-10 illustrate perspective views of a second exemplary system for shaping an implant.

FIG. 11 illustrates an exemplary jig of the system of FIGS. 8-10.

FIG. 12 illustrates an exemplary cutting member of the system of FIGS. 8-11.

FIG. 13 illustrates a flow chart of a method of using the system of FIGS. 8-12.

FIGS. 14-16 illustrate perspective views of a third exemplary system for shaping an implant.

FIGS. 17 and 18 illustrate perspective views of a fourth exemplary system for shaping an implant.

Aspects of the disclosure will now be described in detail with reference to the drawings, wherein like reference numbers refer to like elements throughout, unless specified otherwise.

### DETAILED DESCRIPTION OF ILLUSTRATIVE EMBODIMENTS

The present disclosure provides systems and methods of shaping an implant, such as a bone dowel. The present inventors recognize that it would be favorable for a surgeon to be able to cut or reshape the implant intraoperatively to conform to the specific anatomy of the defect. For example, the present disclosure provides the surgeon systems to reshape a substantially cylindrical shaped blank to form a tapered conical portion to facilitate insertion of the implant and to conform the implant to the anatomical defect. The system may include at least one blade to cut or shave the implant to form the tapered portion. In some embodiments, the shaping may be performed by rotating the implant relative to the at least one blade. The system may include a support shaft and a collar. The implant may have a channel configured to receive the support shaft, and the collar may secure the implant to the support shaft. The support shaft may rotate the implant relative to the at least one blade to form the tapered portion on an outer surface of the implant. Additionally or alternatively, the implant may be received in a sleeve of a jig, and the at least one blade may be inserted into the sleeve to form the tapered portion of the implant. Additional embodiments may include a slot that guides a blade to form the tapered portion of the implant. The systems may be included in a kit including one or more of the implants of various sizes and/or sizing instruments. The system may be used to fill a cavitary defect in a variety of procedures including filling a glenoid during a revision total shoulder arthroplasty.

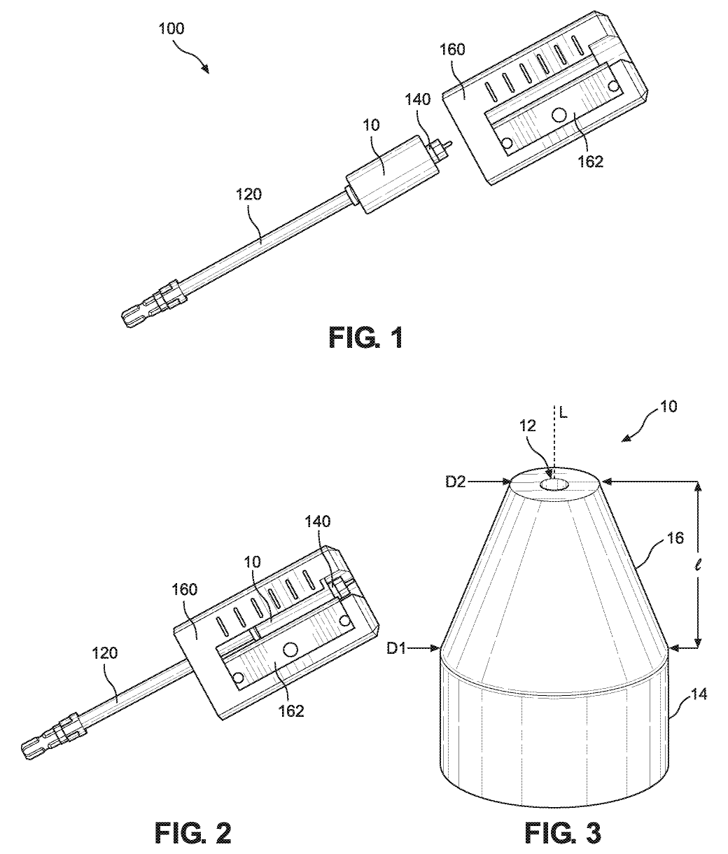

FIGS. 1, 2 and 4-6 illustrate a first exemplary system 100 for shaping an implant 10 according to the present disclosure. The system 100 may include a support shaft 120, a collar 140, and a cutting member 160. As illustrated in FIG. 3, the implant 10 may have a channel 12 configured to receive the support shaft 120. As further illustrated in FIG. 1, the collar 140 may be threaded to a distal end of the support shaft 120 to secure the implant 10 to the support shaft 120. The support shaft 120 may insert the implant 10 into the cutting member 160. The cutting member 160 may have a blade 162, and the support shaft 120 may rotate the implant 10 relative to the blade 162 to shape or cut the implant 10. The system 100 may allow the user to customize the implant 10 based on the location and size of the implant site to be treated.

The implant 10 may have a first pre-formed shape, as illustrated in FIG. 1. The first shape of the implant 10 may include a substantially uniform outer surface along a longitudinal length. The first shape of the implant 10 may have a width or diameter that is substantially uniform along substantially its entire longitudinal length. The first shape of the implant 10 may have a substantially tubular and/or substantially cylindrical cross-section extending substantially its entire longitudinal length. The first shape of the implant 10 may include first and second ends that are rounded and/or tapered. The first shape of the implant 10 may have radial symmetry around a longitudinal axis of the implant 10 and/or lateral symmetry relative to a central lateral axis extending laterally through the longitudinal axis. The first shape of the implant 10 may be considered a pre-formed blank in providing a standard shape that may be cut down into a second customized shape.

FIG. 3 illustrates the second shape of the implant 10 formed by the system 100. The second shape of the implant 10 may have a first portion 14 with a width or diameter D1 that is substantially uniform along substantially the entire longitudinal length of the first portion 14. The width or diameter D1 of the first portion 14 may match the substantially uniform cross-section of the first shape, such that the first portion 14 is not cut by the system 100. The second shape of the implant 10 may have a second portion 16 having a variable cross-section. For example, the second portion 16 may have a truncated conical shape, with a taper extending from a major width or diameter D1 of the first portion 14 to a minor width or diameter D2 at an end of the implant 10. The taper of the second portion 16 may be linear between the major width or diameter D1 and the minor width or diameter D2. In some embodiments, the taper of the second portion 16 may be substantially uniform around a circumference of the implant 10. The minor diameter D2 of the implant 10 may be greater than a diameter of the channel 12. The first portion 14 may form a proximal portion of the implant 10, and the second portion 16 may form a distal portion of the implant 10. The second portion 16 may extend less than an entire length of the implant 10.

The implant 10 may be a bone dowel. The bone dowel 10 may offer surgeons a quick and effective solution for filling bone defects or tunnels during revision surgeries. The bone dowel 10 may provide an immediate structural and/or biologic architecture for stability and incorporation. The bone dowel 10 may be an allograft, made of a porous cancellous bone tissue to provide a scaffold for bony ingrowth and incorporation. The bone dowel 10 may be prewashed to remove native blood and bone marrow elements. The bone dowel 10 may be pre-formed in the first shape in a range of sizes, such as in diameters ranging from 8 mm to 24 mm and longitudinal lengths ranging from 26 mm to 40 mm. The pre-formed diameter may define the major diameter D1 of the second shape after cutting. Before implantation, the bone dowel 10 may be soaked in a platelet-rich plasma (PRP) or bone marrow concentrate to provide an infusion of growth factors and cells to assist with the incorporation of the scaffold.

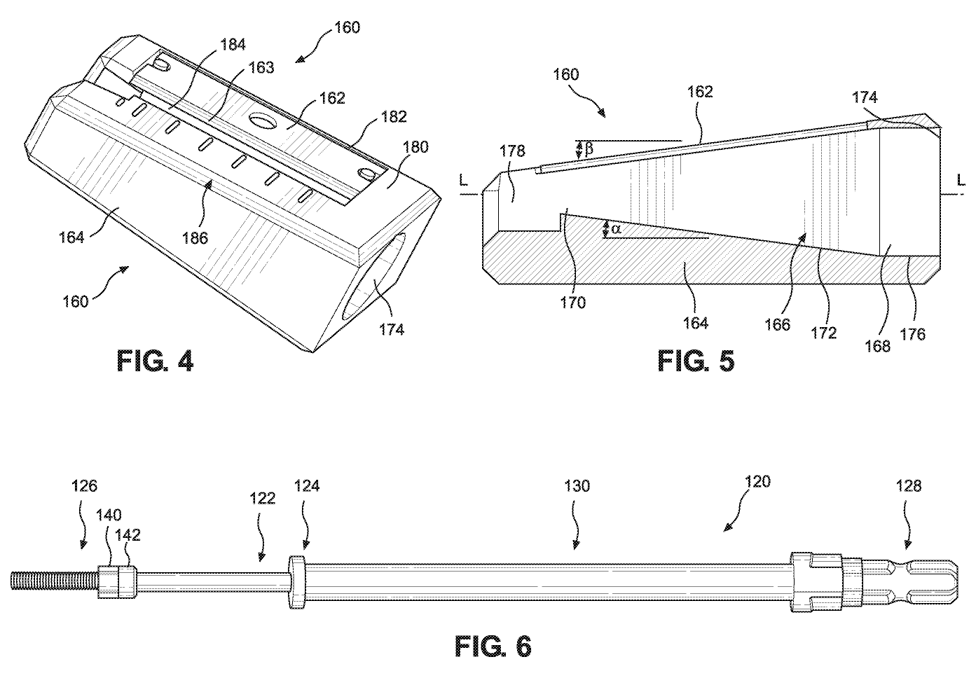

As illustrated in FIGS. 4 and 5, the cutting member 160 may have a housing 164 defining a lumen 166. As further illustrated in the cross-section of FIG. 5, the lumen 166 may have a first portion 168 with a first width or diameter and a second portion 170 with a second width or diameter. The first width or diameter of the first portion 168 may be greater than the second width or diameter of the second portion 170. The housing 164 may have a tapered wall 172 forming the lumen 166 extending between the first portion 168 and the second portion 170. The tapered wall 172 defining the lumen 166 may have a continuous taper between the first portion 168 and the second portion 170 at an angle α relative to the longitudinal axis L. The angle α may be acute. The angle α may be from 1° to 15°, for example from about 5° to about 11°. For example, the angle α may be from about 7° to about 9°. The housing 164 may have a proximal opening 174 leading into the first portion 168 of the lumen 166. The opening 174 may be defined by a substantially cylindrical wall 176 having the first width or diameter. The housing 164 may define a distal portion 178 of the lumen 166 distally of the second portion 170. The distal portion 178 may have a width or diameter greater than the second width or diameter. The distal portion 178 may be configured to receive the collar 140 when the support shaft 120 is inserted into the housing 164. The distal portion 178 may be open at a distal end.

As further illustrated in FIG. 4, the housing 164 may have an upper portion 180 supporting the blade 162. The upper portion 180 may have an opening 182 extending into the lumen 166. The blade 162 may be received in the opening 182 in the upper portion 180 exposing the blade 162 to the lumen 166. The blade 162 may be recessed in the opening 182, such that an upper surface of the blade 162 is substantially flush with an upper surface of the upper portion 180. The blade 162 may extend tangentially of the lumen 166 and define an upper portion of the lumen 166. As illustrated in FIG. 5, the blade 162 may extend at an angle β relatively to the longitudinal axis L to form a substantially uniform tapered cross-section with the tapered wall 172. The angle β may be acute. The angle β may be from 1° to 15°. The angle β may be from about 5° to about 11°. For example, the angle β may be from about 7° or about 9°. The angle β may be the same as the angle α forming a substantially conical cross-section of the lumen 166. The blade 162 may form a linear cut along the angle β to form the taper of the tapered portion 16. The blade 162 may have an edge 163 exposed to the lumen 166 and be configured to cut the implant 10 as the implant 10 is rotated in the lumen 166. The relative rotation may form the taper of the tapered portion 16 substantially uniformly around a circumference of the implant 10. The edge 163 may be sharpened and/or tapered to facilitate the cutting of the implant 10. The edge 163 may be spaced apart from a portion of the housing 164 defining a slot 184 in the cutting member 160. The slot 184 may enable cutting of the implant 10, shedding of the implant shavings, and/or visibility of the implant 10.

The housing 164 may further have indicia 186 on the upper portion 180. The indicia 186 may correspond to the width or diameter of the lumen 166. For example, the indicia 186 may include a plurality of shapes, symbols, and/or numbers along the longitudinal axis L of the housing 164. The indicia may indicate a width or diameter of the lumen 166 at a specific longitudinal position indicated by the indicia 186. Thus, the indicia 186 may include one or more numerical indications and/or tick marks indicating the width or diameter of the lumen 166 at the specific position of the indicia 186. Additionally or alternatively, the indicia 186 may indicate a longitudinal length ℓ of the tapered portion 16 by providing longitudinal length measurements of longitudinal positions of the lumen 166 from the first portion 168. The cutting member 160 may allow the user to customize the implant 10 by rotating and longitudinally advancing the implant 10 relative to the blade 162, where the indicia 186 indicates a minor diameter D2 and/or longitudinal lengths ℓ of the tapered portion 16 based on the longitudinal position of the implant 10 in the lumen 166. The implant 10 may be visible to the user through the slot 184 for the user to visualize the longitudinal position of the distal end of the implant 10. As illustrated in FIG. 4, the indicia 186 may be on the upper portion 180 on an opposite side of the slot 184 as the blade 162. The user may rotate and advance the implant in the housing 164 until the desired minor diameter D2 and/or longitudinal length ℓ is formed based on reading the indicia 186.

The housing 164 may be made of a plastic, such as a high-density polyethylene (HDPE), polypropylene (PP), and/or polyvinyl chloride (PVC), or a metal. The housing 164 may be formed of an integral piece, such as by injection molding. The blade 162 may be made of a metal, such as a stainless steel. The blade 162 may be secured to the housing with a fastener, such as a screw or a bolt.

As illustrated in FIG. 6, the support shaft 120 may have a receiving portion 122, a flange 124, an attachment portion 126, and a fitting 128. The support shaft 120 may be configured to be inserted into the channel 12 of the implant 10, such that the implant 10 is received on the support shaft 120. The implant 10 may be slid on the support shaft 120 until the proximal end of the implant 10 reaches the flange 124. The flange 124 may be proximal of the receiving portion 122 and have a width or diameter greater than the width or diameter of the receiving portion 122. The width or diameter of the flange 124 may be greater than the channel 12 to prevent the flange 124 from passing into the channel 12. The flange 124 may be configured to support the proximal end of the implant 10 and apply a pushing force to advance the implant 10 into the cutting member 160. The flange 124 may have a textured surface and/or teeth configured to grip the implant 10 and prevent rotation of the implant 10 around the longitudinal axis L.

The attachment portion 126 may extend distally from the receiving portion 122 and be configured to engage the collar 140. With the support shaft 120 inserted into the channel 12 of the implant 10, the collar 140 may be attached or slid over the attachment portion 126 to secure the implant 10 on the receiving portion 122. The collar 140 may be configured to prevent the implant 10 from sliding distally off of the receiving portion 122. The collar 140 may also be configured to apply a proximal force against the distal end of the implant 10 to rotationally fix the implant 10 to the support shaft 120. The proximal force of the collar 140 applied to the distal end of the implant 10 may compress the implant 10 between the collar 140 and the flange 124. The compression of the collar 140 may collapse the channel 12 onto the receiving portion 122 to rotationally fix the implant 10 relative to the support shaft 120. A disk 142 may be disposed between the collar 140 and the implant 10. The collar 140 and/or the disk 142 may have a textured surface configured to grip the implant 10 and prevent rotation of the implant 10 around the longitudinal axis L. The textured surface of the collar 140 and/or the disk 142 may include one or more of a surface finish, teeth, a knurl, and/or a spline. The collar 140 may lock the disk 142 in place against the implant 10. The collar 140 and the disk 142 may increase the locking mechanism in a double nut fashion. The collar 140 and the disk 142 may further increase the length of the dowel so that it ends in the threaded portion.

The attachment portion 126 may have an outer thread, the collar 140 and/or the disk 142 may have an inner thread, and the collar 140 may be attached to the attachment portion 126 by rotating the collar 140 and/or the disk 142 relative to the attachment portion 126 to engage the inner thread and the outer thread. However, other mechanical connections may be used to secure the collar 140 and/or the disk 142. For example, the collar 140 and/or the disk 142 may be secured to the attachment portion 126 through a frictional fit, a snap fit and/or a press fit. The mechanical connection may include one or more of a spline, a collet, a leaf spring, and/or a knurl on one or more of the attachment portion 126, the collar 140 and/or the disk 142. The collar 140 and/or the disk 142 may be adjustably attached to the attachment portion 126, for example to accommodate implants 10 of different lengths. The collar 140 and/or the disk 142 may be adjustably attached to the attachment portion 126 through the threaded connection. Additionally or alternatively, the adjustable attachment may be provided by one or more teeth on the attachment portion 126 and/or the collar 140 and/or the disk 142 configured to engage the collar 140 and/or the disk 142 at different longitudinal positions on the attachment portion 126.

The fitting 128 may be configured to be attached to a tool (not shown) to fix or rotate the support shaft 120. The fitting 128 may be a shank of a number of different configurations. The fitting 128 may be a triangular shank, a square shank, a hex shank, a threaded shank, an SDS shank, and/or a morse taper shank. The tool may be a manual or powered tool configured to fix or rotate the implant 10. The tool may be a handle, such as a T handle, for a user to manually apply torque to the support shaft 120. The tool may also be an electric drill having a motor configured to apply torque to the support shaft 120. A shaft portion 130 may extend between the flange 124 and the fitting 128 to impart torque to the receiving portion 122. The shaft portion 130 may have a width or diameter larger than the width or diameter of the receiving portion 122.

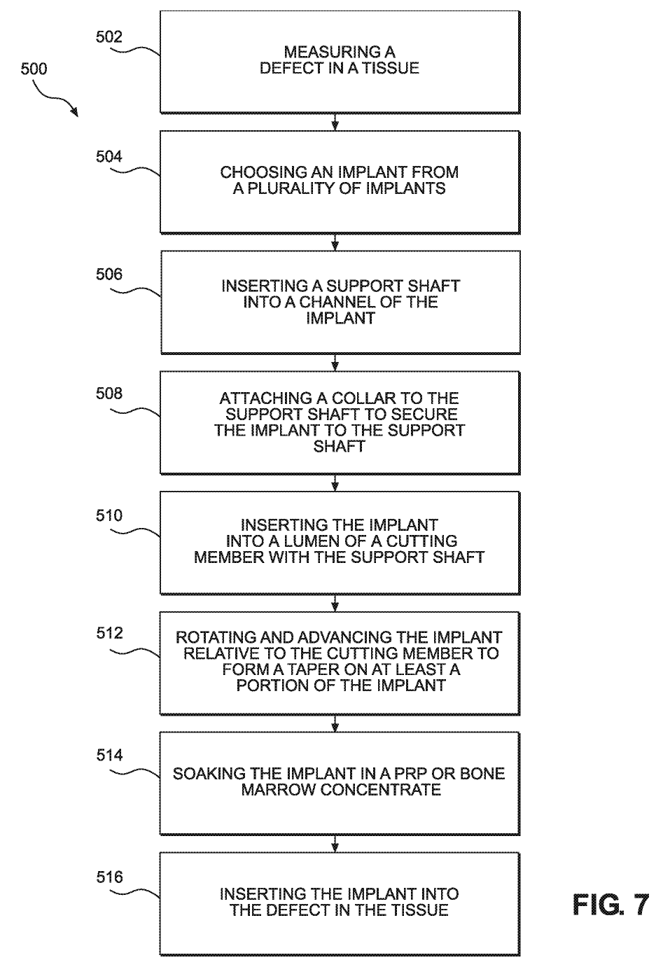

FIG. 7 illustrates a method 500 of using the system 100 to cut the implant 10 and implanting the implant 10. The method 500 may be implemented to customize the implant 10 based on the location and size of the implant sight to be treated. The method 500 may be performed intraoperatively for the user to cut or reshape the implant 10 to conform to specific anatomy of the defect. For example, the user may choose from one of a variety of different implants 10, for example based on a desired material, major diameter D1, and/or total longitudinal length. The user may then customize the implant 10 based on a desired minor diameter D2 and/or longitudinal length ℓ of the tapered portion 16.

In step 502, a defect in a tissue may be measured by a user. The user may measure the depth and/or width of the defect visually to estimate the depth and/or width. The depth of the defect may be measured by insertion of a graduated wire into the defect, and the graduations may indicate a depth of the defect. The width of the defect may be measured by a caliper configured to measure the width of an opening of the defect. Additionally or alternatively, the width of the defect may be measured by inserting a hole gauge having an adjustable distal end configured to expand to the width of the defect and an output with an indicator of the width. The defect may also be measured through trial and error. For example, implants 10 of different sizes may be inserted into the defect to measure the depth and/or width of the defect for a desired implant.

In step 504, an implant 10 may be chosen by the user from a plurality of the implants 10. A plurality of the implants 10 may be provided in a kit, and the user may choose an implant 10 of a size for best fit into the defect. The implants 10 may have a variety of diameters and/or lengths. For example, the implants 10 may have diameters ranging from 8 mm, 10 mm, 12 mm, and/or 14 mm and/or longitudinal lengths of 26 mm, 26 mm, 34 mm, and/or 40 mm. The user may choose the implant 10 having the diameter and/or length to best fill the defect. The user may then customize the implant 10 based on a desired minor diameter D2 and/or longitudinal length ℓ of the tapered portion 16. The implant 10 may initially have a substantially cylindrical shape having the major diameter D1.

In step 506, the implant 10 may be attached to the support shaft 120 by inserting the support shaft 120 into the channel 12 of the implant 10. The implant 10 may be slid onto the receiving portion 122 until the proximal end of the implant 10 contacts the flange 124. The flange 124 may have a textured surface and/or teeth configured to grip the proximal end of the implant 10.

In step 508, the implant 10 may be secured to the support shaft 120 by the user attaching the collar 140 to the attachment portion 126 of the support shaft 120. The collar 140 may apply a proximal force to the disk 142 to compress the implant 10 and rotationally fix the implant 10 to the support shaft 120. The disk 142 may have a textured surface and/or teeth configured to grip the distal end of the implant 10. The collar 140 may be secured to the attachment portion 126, for example by a threaded connection.

In step 510, the implant 10 may be inserted through the opening 174 into the lumen 166 of the cutting member 160 by the user with the support shaft 120.

In step 512, the support shaft 120 may rotate and advance the implant 10 relative to the cutting member 160 to form a taper on at least a portion 16 of the implant 10. The tapered portion 16 may be formed by rotating the implant 10 relative to the blade 162 to cut or shave a peripheral portion of the cylindrical cross-section to form the tapered portion 16. The support shaft 120 may further advance the implant 10 into the lumen 166. Advancing the implant 10 may reduce the minor diameter D2 of the implant 10 and lengthen the tapered portion 16 along the longitudinal axis of the implant 10. The user may customize the implant 10 by advancing the implant 10 into the lumen 166 a distance to form the tapered portion 16 at a specific longitudinal length ℓ. The user may directly visualize the relative position of the implant 10 in the cutting member 160 through the slot 184. The user may determine the corresponding minor diameter D2 of the implant 10 based on the longitudinal position of the implant based on the indicia 186 on the housing 164. Additionally or alternatively, the user may determine the longitudinal length ℓ of the tapered portion 16 based on the longitudinal position of the implant and the indicia 186 on the housing 164. The user may form the implant 10 for customization based on the location and size of the defect to be treated.

Step 512 may be performed by rotating and advancing the support shaft 120 for example with the tool via the fitting 128 and/or manually holding or fixing the cutting member 160. Additionally or alternatively, the relative rotation of the implant 10 may be provided by rotating and advancing the cutting member 160 and/or holding or fixing the support shaft 120 with the tool via the fitting 128. After cutting the implant 10, the support shaft 120 may be retracted from the cutting member 160 to remove the implant 10 from the lumen 166 of the cutting member 160. The collar 140 and the disk 142 may be removed from the support shaft 120, and the implant 10 may be removed from the support shaft 120.

In step 514, the implant 10 may be soaked in a PRP or bone marrow concentrate. The soaking may provide an infusion of growth factors and cells to assist with the incorporation of the scaffold of the implant 10.

In step 516, the implant 10 may be inserted by the user into the defect. The implant 10 may be inserted over a guide pin, such that the guide pin is received in the channel 12. The implant 10 may be positioned in a delivery sleeve. The implant 10 may be advanced into the defect, with the second tapered portion 16 leading the first cylindrical portion 14. The implant 10 may be particularly suitable during a revision total shoulder arthroplasty in filling a glenoid defect, which is often conical. The tapered portion 16 may substantially match the conical shape of the glenoid defect. In some embodiments, additional implants 10 and/or shavings from cutting of the implant 10 may be added to the defect to substantially fill the entire defect.

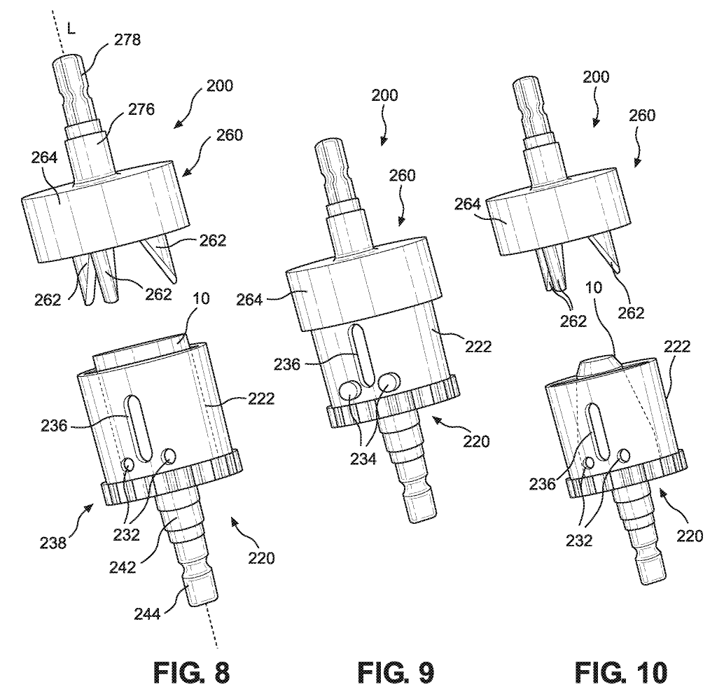

FIGS. 8-12 illustrate a second exemplary system 200 for cutting the implant 10 according to the present disclosure. The system 200 may include a jig 220 and a cutting member 260. The jig 220 may have a sleeve 222 configured to receive the implant 10. The cutting member 260 may have at least one blade 262. For example, the cutting member 260 may have a plurality of blades 262 circumferentially spaced around a longitudinal axis L of the cutting member 260. The sleeve 222 of the jig 220 may be configured to receive the at least one blade 262. The implant 10 may be rotated and advanced relative to the at least one blade 262 to cut the implant 10 to form the tapered portion 16. The system 200 may allow the user to customize the implant 10 based on the location and size of the defect to be treated, as discussed with reference to the system 100, the entire disclosure of which is incorporated herein for sake of brevity.

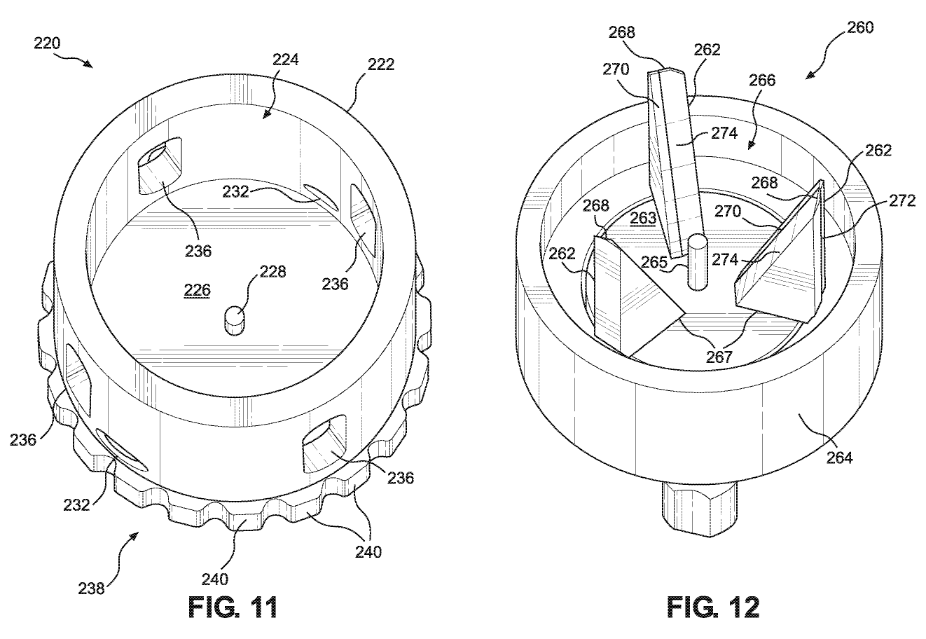

As illustrated in FIGS. 8 and 11, the sleeve 222 of the jig 220 may have a lumen 224 configured to receive the implant 10 with the first shape. The sleeve 222 may extend distally from a proximal wall 226 that defines a closed proximal end of the lumen 224. The proximal wall 226 may be configured to provide a backstop and support the proximal end of the implant 10. A projection 228 may extend distally from the proximal wall 226 and be configured to be received in the channel 12 of the implant 10 to centrally align the implant 10 in the sleeve 222. The projection 228 may be substantially cylindrical and have a width or diameter corresponding to the channel 12 of the implant 10. The sleeve 222 may have at least one hole 232 therethrough configured to receive at least one pin 234. The at least one pin 234 may be inserted through the at least one hole 232 to penetrate or compress the implant 10 to fix or secure the implant 10 to the jig 220 rotationally and longitudinally. For example, the sleeve 222 may have a plurality of holes 232, each configured to receive a pin 234. Additionally or alternatively, the implant 10 may be secured to the sleeve 222 through one or more of a frictional fit, a snap fit and/or a press fit. The sleeve 222 may have one or more of an internal spline, an internal collet, an internal leaf spring, and/or an towel clamp configured to secure the implant 10 therein. The sleeve 222 may have at least one slot 236 therethrough configured to allow for visibility of the implant 10 and/or the at least one blade 262 when inserted into the jig 220. The at least one slot 236 may be elongated to allow for visibility of the tapered portion 16 being formed. Additionally or alternatively, the at least one slot 236 may be configured for material removal, evacuation, and/or cleaning of the sleeve 222. Additionally or alternatively, the at least one slot 236 may be configured to receive one or more pins 234 (as discussed above). The at least one slot 236 may have a length along the longitudinal axis of the sleeve 222 and a width extending circumferentially, where the length is greater than the width. The at least one slot 236 may include a plurality of slots 236 circumferentially disposed around the sleeve 222, as illustrated. The sleeve 222 may have a circumferential surface 238 on a proximal portion that is textured to facilitate gripping by the user. For example, the circumferential surface 238 may have a plurality of teeth or castellations 240 extending circumferentially around the longitudinal axis L of the sleeve 222. A shaft 242 having a fitting 244 may extend proximally from the proximal wall 226 of the sleeve 222. The fitting 244 may be configured to be attached to a tool (not shown) to fix or rotate the jig 220, as discussed with reference to the fitting 128, the entire discussion is incorporated herein for sake of brevity.

As further illustrated in FIG. 12, the cutting member 260 may have a sleeve 264 defining a lumen 266. The sleeve 264 may extend proximally from a distal wall 263 defining a closed distal end of the lumen 266. A projection 265 may extend proximally from the distal wall 263 and be configured to be received in the channel 12 of the implant 10 to centrally align the implant 10 in the sleeve 264. The projection 265 may be substantially cylindrical and have a width or diameter corresponding to the channel 12 of the implant 10. The projection 265 may have a length greater than a length of the projection 228 of the jig 220. The sleeve 264 may have an inner diameter larger than an outer diameter of the sleeve 222 of the jig 220. The sleeve 264 of the cutting member 260 may receive the sleeve 222 of the jig 220 in the lumen 266 along the longitudinal axis L, while the at least one blade 262 is inserted into the lumen 224 of the jig 220. The concentric insertion of the sleeve 222 of the jig 220 in the sleeve 264 of the cutting member 260 may rotationally stabilize the cutting member 260 relative to the jig 220 along the longitudinal axis L. The at least one blade 262 may be configured to rotate and advance into the lumen 224 of the jig 220 cutting or shaping the implant 10. The at least one blade 262 may be configured to advance until the distal end of the sleeve 222 of the jig 220 contacts the distal wall 263 of the cutting member 260. The minor diameter D2 and the length ℓ of the tapered portion 16 may be based on the amount of advancement of the at least one blade 262 into the sleeve 222 of the jig 220. Similar to the system 100, the system 200 may have indicia (not shown) indicating the resultant minor diameter D2 and/or the length ℓ from the advancement of the cutting member 260. For example, the indicia may be a graduated measurement on an outer surface of the sleeve 222 of the jig 220 that is selectively covered by the advancement of the sleeve 264 of the cutting member 260. The resultant minor diameter D2 and/or the length ℓ may be indicated by the indicia that is exposed or not covered by the sleeve 264 of the cutting member 260.

The sleeve 264 may receive at least a portion of the at least one blade 262. The at least one blade 262 may have a distal end 267 fixed to the distal wall 263 and extend proximally through the lumen 266. The at least one blade 262 may have a proximal end 268 extending out of the lumen 266 and being exposed proximally from the sleeve 264. The proximal end 268 may form a sharp tip defined by the convergence of an inner surface 270 and an outer surface 272. The outer surface 272 may extend along the longitudinal axis L of the cutting member 260, and the inner surface 270 may extend at an angle relative to the longitudinal axis L of the cutting member 260. The angle of the inner surface 270 may be acuate. The angle of the inner surface 270 may be from 0° to 15° relative to the longitudinal axis L of the cutting member 260. For example, the angle of the inner surface 270 may be from about 8° to about 13°. For example, the angle of the inner surface 270 may be from about 10° to about 11°. The inner surface 270 may have a face 274 extending at an angle relative to a lateral axis of the at least one blade 262. The face 274 may be on the same rotational direction of the plurality of blades 262, such as on a clockwise side of the blades 262. In use, the distal end 268 of the at least one blade 262 may engage the implant 10, and the inner surface 270 of the at least blade 262 may cut the circumference of the implant 10 to form the tapered portion 16. The face 274 may provide a cut relief and extend at an angle of the inner surface 270 at about 15° from the inner surface 270. As illustrated in FIG. 8, a shaft 276 having a fitting 278 may extend distally from the distal wall 263 of the cutting member 260. The fitting 278 may be configured to be attached to a tool (not shown) to fix or rotate the cutting member 260, as discussed with reference to the fitting 128 and/or 244, the entire discussion is incorporated herein for sake of brevity.

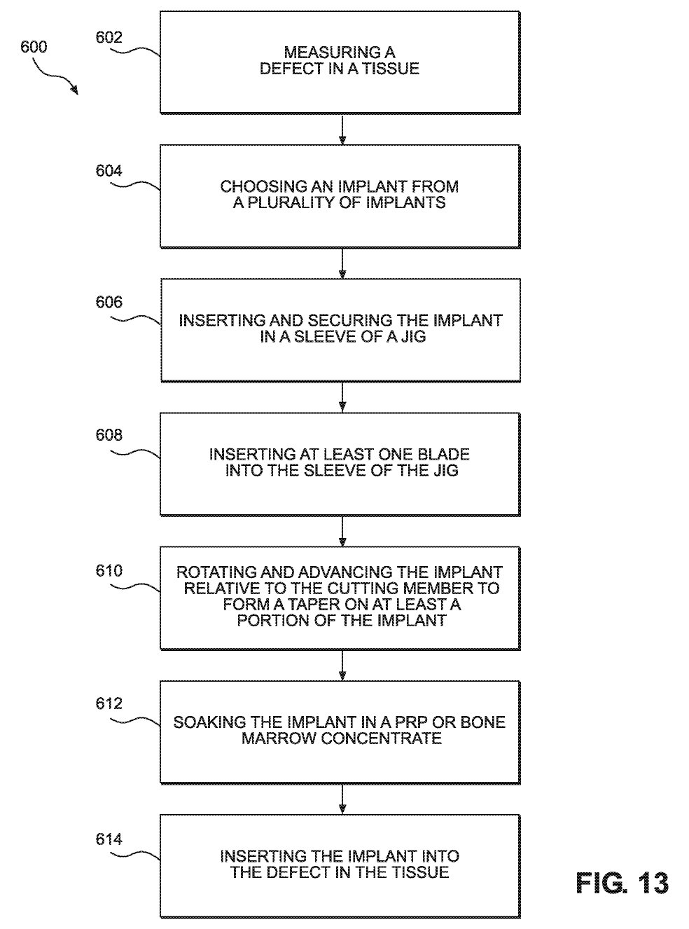

FIG. 13 illustrates a method 600 of using the system 200 to cut the implant 10. The method 600 may be implemented to customize the implant 10 based on the location and size of the implant sight to be treated. For example, the user may choose from one of a variety of different implants 10, for example based on a desired material, major diameter D1, and/or longitudinal length. The user may then customize the implant 10 based on a desired minor diameter D2 and/or longitudinal length ℓ of the tapered portion 16. The method 600 may be performed similar to the method 500, the entire disclosure is incorporated for sake of brevity.

For example, as discussed with reference to the method 500, in step 602, a defect in a tissue may be measured by a user, and in step 604, an implant 10 may be chosen by the user from a plurality of the implants 10.

In step 606, the implant 10 may be inserted and secured in the sleeve 222 of the jig 220. The implant 10 may be secured or fixed in the sleeve 222 with the one or more pins 234 inserted in the one or more holes 232. The one or more pins 234 may compress and/or frictionally engage the implant 10 to secure the implant 10 translationally and rotationally relative to the jig 220.

In step 608, the at least one blade 262 may be inserted into the sleeve 222 of the jig 220 by approximating the jig 220 and the cutting member 260 along the longitudinal axis L. The sleeve 222 of the jig 220 may be inserted in the sleeve 264 of the cutting member 260 to rotationally stabilize the cutting member 260 relative to the jig 220 along the longitudinal axis L.

In step 610, the jig 220 may rotate and advance the implant 10 relative to the cutting member 260 to form the tapered portion 16 of the implant 10. The inner surface 270 of the at least one blade 262 may shape the tapered portion 16 of the implant 10. The cutting member 260 may be advanced relative to the jig 220 a determined distance to customize the minor diameter D2 and/or longitudinal length ℓ according to the defect intended to be treated. The relative rotation may be formed by rotating at least one of the jig 220 and/or the cutting member 260 relative to each other about the longitudinal axis L.

As discussed with reference to the method 500, in step 612, the implant 10 may be soaked in a PRP or bone marrow concentrate, and in step 614, the implant may be inserted by the user into the defect.

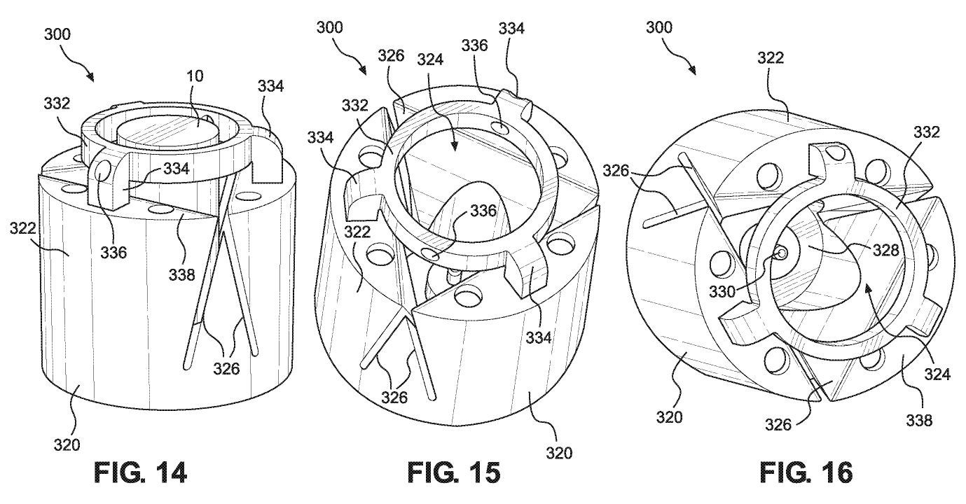

FIGS. 14-16 illustrate a third exemplary system 300 for forming the implant 10 according to the present disclosure. The system 300 may include a jig 320. The jig 320 may have a sleeve 322 with a lumen 324 configured to receive the implant 10. The jig 320 may have at least one slot 326 extending through the sleeve 322. The at least one slot 326 may be configured to receive a blade (not shown) to cut the implant 10 to form the tapered portion 16. The system 300 may allow the user to customize the implant 10 based on the location and/or size of the implant sight to be treated, as discussed with reference to the systems 100, 200, the entire disclosure of which is incorporated herein for sake of brevity.

The sleeve 322 may have a distal wall 328 defining a closed distal end of the lumen 324. The distal wall 328 may be configured to provide a backstop and support the proximal end of the implant 10. A projection 330 may extend proximally from the distal wall 328 and be configured to be received in the channel 12 of the implant 10 to centrally align the implant 10 in the sleeve 322. The projection 330 may be substantially cylindrical and have a width or diameter corresponding to the channel 12 of the implant 10. The implant 10 may extend proximally out of the sleeve 322 and be received in a collar 332 at a proximal portion of the jig 320. The collar 332 may have an outer diameter less an outer diameter of the sleeve 322. The collar 332 may be attached to the sleeve 322 by a plurality of supports 334 circumferentially spaced apart around a longitudinal axis of the jig 320. For example, the collar 332 may be attached to the sleeve 322 with three supports 334 evenly spaced circumferentially the longitudinal axis of the jig 320. The spacing of the collar 332 and supports 334 may provide visibility of the implant 10 in the jig 320. The supports 334 may be disposed on an outer surface of the collar 332. The collar 332 and/or supports 334 may have at least one hole 336 configured to receive a pin (234, as illustrated in FIG. 9) to secure or fix the proximal portion of the implant 10 to the jig 320. For example, as illustrated, the jig 320 may have a pair of holes 336 on opposing sides of the longitudinal axis, one of the holes 336 being through the collar 332 and a support 334 and one of the holes 336 only through the collar 332.

The at least one slot 326 may define opposing surfaces defined by the sleeve 322 configured to receive the blade therebetween. The at least one slot 326 may extend from an upper surface 338 of the sleeve 322 to the distal wall 328. The blade may be inserted into the at least one slot 326 at an upper portion of the sleeve 322 below or distal of the collar 332. The at least one slot 326 may intersect the lumen 324 for the inserted blade to form the tapered portion 16 in the implant 10. The least one slot 326 may extend through opposing segments of the outer surface of the sleeve 322, such that the blade may extend laterally, completely through a cross-section of the sleeve 322. The at least one slot 326 may extend through the sleeve 322 at an angle relative to the longitudinal axis of the jig 320. The angle of the at least one slot 326 may be acute. The angle may be from 5° to 25° relative to the longitudinal axis L of the jig 320. For example, the angle may be from about 10° to about 15° relative to the longitudinal axis L of the jig 320. As illustrated, the at least one slot 326 may include a plurality of slots 326 around the circumference of the sleeve 322. For example, the sleeve 322 may have three slots 326 around the circumference of the jig 320. Edges of adjacent slots 326 may intersect at the upper surface 338 of the sleeve 322 and diverge along the longitudinal axis of the sleeve 322, as illustrated.

The system 300 may be used in a method similar to the methods 500 and/or 600, as incorporated herein for sake of brevity. The implant 10 may be inserted into the sleeve 322 of the jig 320 and fixed with the at least one pin. A blade may be inserted at the upper portion of each of slot 326 and passed through the slot 326 to cut the implant 10 to form the tapered portion 16. In the example of the jig 320 having three slots 326, the system 300 may form three facets of the tapered portion 16. After the three facets are formed, the implant can be released from the pins and rotated in the lumen 324. The blade may be inserted and advanced in the slots 326 again to form additional facets. The rotation and cutting may be repeated any number of times to form a more rounded and uniform cross-section of the tapered potion 16.

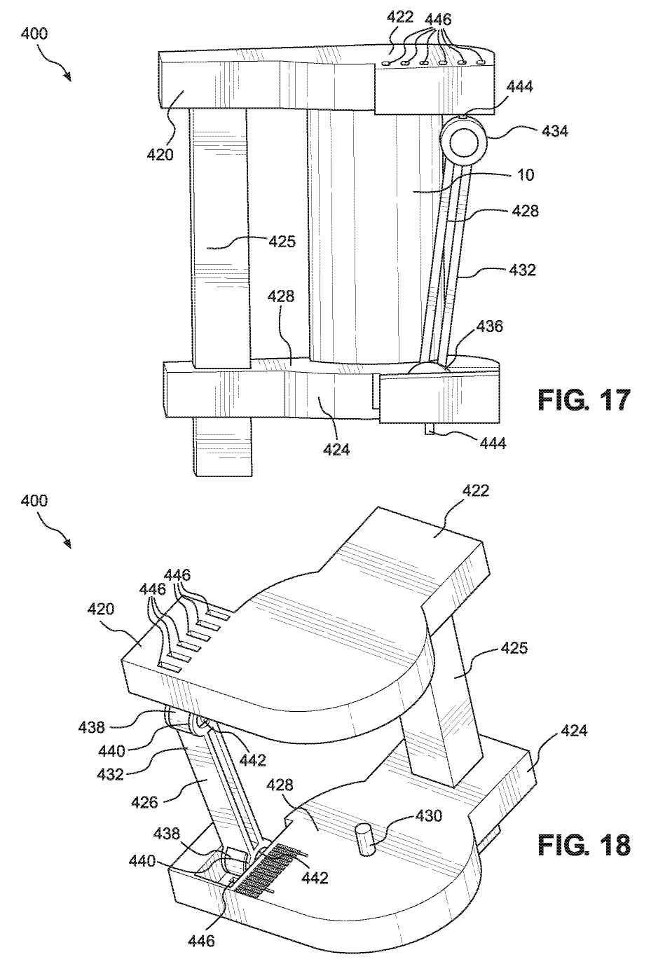

FIGS. 17 and 18 illustrate a fourth exemplary system 400 for cutting the implant 10 according to the present disclosure. The system 400 may include a jig 420. The jig 420 may have a proximal housing member 422 and a distal housing member 424 configured to receive the implant 10. The jig 420 may include a guide 426 extending between the proximal housing member 422 and the distal housing member 424. The guide 426 may have a slot 428 configured to receive a blade (not shown) to cut the implant 10 and form the tapered portion 16. The system 400 may allow the user to customize the implant 10 based on the location and size of the implant sight to be treated, as discussed with reference to the systems 100, 200, 300, the entire disclosure of which is incorporated herein for sake of brevity.

The proximal housing member 422 and the distal housing member 424 may be releasably attached and/or separable to receive the implant 10 therebetween. The proximal housing member 422 may have a shaft 425 configured to be received through and opening of the second housing member 424. The distal housing member 424 may be configured to slide along the shaft 425 to adjust a spacing between the distal housing member 424 and the proximal housing member 422 to receive the implant 10 therebetween. Each of the housing members 422, 424 may have an inner surface 429 with a protrusion 430 configured to be inserted into the channel 12.

The guide 426 may be adjustably positioned between the proximal housing member 422 and the distal housing member 424 to vary the angle of the tapered portion 16 relative to the longitudinal axis L. The guide 426 may have a swivel connection to each of the proximal housing member 422 and the distal housing member 424 to adjust for different diameters of the implant 10 and/or angles of taper. The guide 426 may include a central strut 432 defining the slot 428, a proximal connector 434 connected to the proximal housing member 422, and a distal connector 436 connected to the distal housing member 424. The ends of the central strut 432 may be configured to rotate or swivel relative to the proximal connector 434 and the distal connector 436, respectively. For example, the proximal connector 434 may have a collar 438, the proximal portion of the central strut 432 may have a collar 440, where each of the collars receive and may be configured to rotate around an axis of a proximal bearing 442. Similarly, the distal connector 436 may have a collar 438, the distal portion of the central strut 432 may have a collar 440, where each of the collars 438, 440 may receive and be configured to rotate around an axis of a distal bearing 442. Each of the connectors 434, 436 may have a protrusion 444 selectively received in one of a plurality slots 446 in each of the housing members 422, 424 to adjust the angle of the taper. The position of each protrusion 444 in the respective slot may define a position of the connector 434, 436, and the ends of the central strut 432 may accordingly rotate relative to each of the connectors 434, 436 to define a cutting angle for the tapered portion 16.

The system 400 may be used in a method similar to the methods 500 and/or 600, as incorporated herein for sake of brevity. The implant 10 may be inserted between the housing members 422, 424. The guide 426 may be adjusted to achieve the desired cutting angle. A blade may be inserted through the slot 428 to form the tapered portion 16. Similar to the third system 300, rotation and cutting may be repeated any number of times to form a faceted and/or rounded cross-section of the tapered potion 16.

The systems 100, 200, 300, 400 may constitute a kit according to the present invention. The kit may include components of one or more of the systems 100, 200, 300, 400. The kits may include different sizes or configurations. For example, the kit may include a plurality of different cutting members 160, 260 of different angles β, major diameters D1, and/or minor diameters D2. The kit may include a plurality of the implants 10 of various sizes, as discussed herein. For example, the kit may include a first implant 10 and a second implant 10, the first implant 10 may have a first dimension, the second implant 10 may have a second dimension, and the first dimension may be greater than the second dimension. The dimension may be a length and/or a major diameter D1. The kit may include additional implants 10, including a third implant 10, a fourth implant 10, a fifth implant 10, and/or a sixth implant 10 (and so forth) of the same and/or different dimensions (e.g., length and/or diameter). At least one or all of the implants 10 may be an allograft, such as a porous cancellous bone tissue. The kit may further include one or more sizing instruments, such as a graduated wire, a caliper, and/or a hole gauge. The graduated wire may be configured to be inserted into the defect to measure a depth of the defect. The caliper may be configured to measure a width of the defect. The hole gauge may have an adjustable distal end configured to expand to the width of the defect and an output with an indicator of the width. The kit may include one or more reamers for adjusting the size of the defect. The kit may further include a tamp and/or a guide pin configured to allow for placement of the implant 10 into bone tunnels. Using allograft bone dowels to fill bone defects allows for proper fitting of the implant 10 and immediate structural support bone tunnels and cystic bone areas.

It will also be appreciated by those skilled in the art that modifications can be made to the example embodiments described herein without departing from the invention. Structural features of systems and apparatuses described herein can be replaced with functionally equivalent parts or omitted entirely. Moreover, it will be appreciated that features from the embodiments can be combined with each other without departing from the disclosure.

What is claimed is:

1. A system for cutting an implant, the system comprising:
a support shaft configured to be passed through a channel of the implant;
a collar configured to be attached to the support shaft to secure the implant to the support shaft; and
a cutting member having a blade and a lumen configured to receive the implant, wherein the support shaft is configured to rotate the implant relative to the blade to form a taper on at least a portion of an outer surface of the implant.

2. The system of claim 1, wherein the blade extends at an acute angle relative to a longitudinal axis of the lumen.

3. The system of claim 2, the acute angle is from 5° to 15°.

4. The system of claim 1, wherein the blade extends tangentially of the lumen.

5. The system of claim 1, wherein the lumen has a first opening with a first width and a second opening with a second width, and the first width is greater than the second width.

6. The system of claim 1, wherein the lumen has a tapered cross-section.

7. The system of claim 1, wherein the cutting member has indicia indicating a width of the lumen.

8. The system of claim 1, wherein:
the cutting member has a housing defining the lumen and an opening that receives the blade,
the blade is fixed to the housing, and
an edge of the blade is exposed to the lumen.

9. The system of claim 8, wherein the cutting member has a longitudinal slot between the housing and the blade.

10. The system of claim 1, wherein:
the support shaft has a receiving portion configured to be inserted into the channel of the implant, and a flange proximal of the receiving portion, and
the flange is configured to support a proximal portion of the implant.

11. The system of claim 1, wherein the collar is configured to be adjustably attached to the support shaft.

12. The system of claim 1, wherein:
the support shaft has a distal portion with an outer thread,
the collar has an inner thread, and
the outer thread is configured to engage the inner thread to attach the collar to the support shaft.

13. The system of claim 1, wherein the support shaft has a proximal portion having a fitting configured to be attached to a tool.

14. A kit comprising:
at least one implant; and
the system of claim 1.

15. The kit of claim 14, wherein the at least one implant is an allograft.

16. The kit of claim 14, wherein the at least one implant is a porous cancellous bone tissue.

17. A method comprising:
attaching an implant to a support shaft;
inserting the implant into a cutting member, the cutting member having a blade extending at an acute angle relative to a longitudinal axis of the cutting member; and
rotating the support shaft relative to the cutting member to form a taper on at least a portion of an outer surface of the implant.

18. The method of claim 17, further comprising advancing the support shaft along the longitudinal axis of the cutting member while the rotating of the support shaft relative to the cutting member.

19. The method of claim 17, wherein the taper is formed substantially uniformly around a circumference of the implant.

20. The method of claim 17, wherein the taper is formed on less than an entire length of the implant.
